(19) United States
(12) Patent Application Publication
(10) Pub. No.: US 2024/0423641 A1
(43) Pub. Date: Dec. 26, 2024

(54) SHAPING INSTRUMENT

(71) Applicant: THE AUSTRALIAN ON-LINE PROSTHETIC COMPANY PTY LTD, BURNIE (AU)

(72) Inventor: SCOTT FLETCHER, BURNIE (AU)

(73) Assignee: THE AUSTRALIAN ON-LINE PROSTHETIC COMPANY PTY LTD, BURNIE (AU)

(21) Appl. No.: 18/829,413

(22) Filed: Sep. 10, 2024

(30) Foreign Application Priority Data
Mar. 11, 2022 (WO) PCT/AU2022/050211

(51) Int. Cl. A61B 17/16; A61B 17/00

(52) U.S. Cl. CPC A61B 17/1613; A61B 2017/00477

(57) ABSTRACT

A shaping instrument for the preparation of a surface of a work body utilizes regional or a combination of local and regional movement provided by a rotational drive and one or more transfer mechanism. The shaping instrument includes a contoured body having engagement elements on an active surface to work a passive surface of a work body, and a transfer mechanism connectable to a rotatable drive and engageable with the contoured body. The transfer mechanism converts the rotational movement of the rotatable drive to at least one small local movements of one or more parts of the contoured body or a circumferential regional movement of the same parts around a different (longitudinal) axis. The shaping instrument is able to work the passive surface of the work body by one or more of these two movements.

### CROSS REFERENCE TO RELATED CO-PENDING APPLICATIONS

This application is a continuation-in-part and claims the benefit of International PCT application Serial No. PCT/AU2022/050211 filed on Mar. 11, 2022 and entitled “Shaping Instrument”, which is commonly assigned and the contents of which are expressly incorporated herein by reference.

### FIELD OF THE INVENTION

The present invention relates to a shaping instrument. The instrument is for working of a work body. This can relate to woodwork, plaster, bone or a range of other worked materials. The shaping instrument can be driven by handheld power tools.

In a particularly preferred embodiment, the invention relates to instruments for preparing a bone during surgical procedures. It also relates to prostheses including preparation of bone for attaching prostheses and improvements in the prostheses itself.

The invention is described as it applies to hip arthroplasty but has application to a range of surgical procedures requiring bone contouring and shaping, with or without the need for definitive structural support or the need to use a prosthesis. In a more general sense, the invention can be applied to contouring any adjacent or co-located material/s. The application, for example, could include the underground tunnelling to layout subterranean pipes or shaping wood.

### BACKGROUND

In manually working materials there is a tendency to use large swiping actions. Further the instruments usually have a cutting edge or working edge on one side (alone). This results in the device being used solely in one direction. Further due to the right-handedness or left-handedness of the user, the user will have a tendency to gouge in one direction greater than the other. These limitations will mean that the prior art instruments will not provide an even symmetrical working but will provide a contoured surface of lesser surface quality.

If those manual handling techniques are driven by handheld power tools, then such a tool can be a power drill. This could be a do-it-yourself tool or could be a precision medical tool.

When applied to the art of hip arthroplasty, the contouring instrument uses a combination of rapid local oscillation movements together with regional circumferential movements to shape or contour the acetabulum with or without embedding the instrument into the acetabular bone for use as structural support or definitive prosthesis.

Other examples of surgeries where the present art could be employed include, but are not limited to, the shaping, contouring, structural support or application of a prosthesis to the femoral head during hip resurfacing procedures, to the glenoid during shoulder arthroplasty, to the intra-medullary preparation of long bones required prior to the insertion of intra-medullary rods or nails. The invention could be applied to shaping a bone defect prior to structural stabilization within a single bone or between two adjacent bones.

Prior art describes bone reamers used to prepare bone surfaces during hip arthroplasty and other bone surgeries. Prior art one reamers are varied in their surface designs (U.S. Pat. No. 968,188(B) and housing (CN 201822888U, CN202078365U).

Some designs are “minimally invasive”, limited use (US20130204254A1) or disposable (EP2957245B1) and have drive shaft that are offset from the direction of the acetabular reaming (US 20150320428A1). All have a traditional circular unidirectional rotational movement of the bone reamer. The reamer is usually rotated in a clockwise direction and the cutting aspects of the reamer are manufactured to only cut bone when the reamer in moving in this clockwise direction.

Bone reamers described in prior art using large fast-moving circular (uni-directional) excursions of exposed sharp cutting edges to shape or contour bone can expose the patient to safety risks. Such risks include sweeping, tearing and cutting of soft tissue adjacent to the target bone.

Large fast-moving excursions of the rotating cutting shell can result in the sudden transfer of large (moment) forces to the operator's hand and wrist, should the rotating element shell suddenly jam at the shell/bone interface.

Prior art describes rapid oscillation bone cutting saws (U.S. Pat. No. 5,092,869A). Such saws can be oscillating or reciprocating and are used for bone resection and are used to resect or cut bone in a two-dimensional way. Rapid oscillation bone instruments have not been used as surface sculpting tools in bone surgery.

Prior art is silent on the application of contouring, shaping instruments as definitive structural supports or definitive prostheses.

A secondary purpose of the invention is to use the oscillating instrument as a structural component within the work body. This structural support can be incorporated into a definitive prosthetic component.

When performing hip arthroplasty, the acetabulum must be prepared to a precise depth and position so as to respect the structure and function of the surrounding hip soft tissue envelope.

It is important that the depth of resection of the acetabular bone (the “floor” of the acetabulum) is not over-reamed or under reamed to the unplanned acetabular depth.

Mal-position of the acetabular component may result in altered bio mechanical function of the hip replacement leading to complications such as hip instability, bone impingement of prosthesis and early prosthesis failure.

Precision in hip component positioning is associated with improved patient outcomes in hip arthroplasty.

The adoption of minimally surgery has made it more difficult to directly visualize the placement of acetabular components to a precise position and with a precise depth.

Surgeons have sought to use advanced technology in order to improve accuracy of acetabular component placement. Such advanced technology used to assist accuracy of acetabular placement includes computer navigation, robotics, patient specific guides, intra-operative radiology (imaging) and three axis digital accelerometer instruments.

The costs associated with the use of new technology has prohibited the routine adoption of these technologies when costs are contained.

There is an opportunity to improve conventional instrumentation in order to control depth and direction of the shaping of the acetabulum.

The present invention seeks to provide Oscillating Bone Reamer/Prosthesis, which will overcome or substantially ameliorate at least one or more of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

In accordance with the invention there is provided a shaping instrument using regional or a combination of local and regional movement provided by a rotational drive means and one or more transfer means.

A shaping instrument for the preparation of a surface of a work body including:
a. a contoured body having engagement elements on an active surface to work a passive surface of a work body;
b. a transfer mechanism connectable to a rotatable drive and engageable with the contoured body;
c. wherein the transfer mechanism converts the rotational movement of the rotatable drive to at least one small local movements of one or more parts of the contoured body or a circumferential regional movement of the same parts around a different (longitudinal) axis; and
d. wherein the shaping instrument is able to work the passive surface of the work body by one or more of these two movements.

A shaping instrument using a movement of local and/or regional only movement.

A shaping instrument using on regional only movement with depth control.

A shaping instrument wherein the contoured body includes a non-cutting portion.

A shaping instrument wherein the non-cutting portion of the contoured body is located at a polar top of the contoured body.

A shaping instrument wherein the non-cutting portion of the contoured body of the shaping instrument is without a polar cutting section, and will result in controlled localized contouring around the engagement elements in only the non-polar region.

A shaping instrument wherein the non-cutting portion regulates the direction and depth of bone resection.

A shaping instrument according to claim 1 wherein the rotational movement of a drive shaft provides total rotational movement of the whole work body surface of the contoured curved body.

A shaping instrument wherein the shaping of a work body is a one or two stage shaping process.

A shaping instrument wherein the shaping of a work body is a two-stage shaping process. wherein each of the two-step process is applied at a different depth.

A shaping instrument wherein the two-stage or multi-stage process is undertaken with different shaped and depth shaping instruments to give control to differing locations and depths on the work body.

A shaping instrument wherein different curvatures of the shaping instruments are used in different combinations to effect different effect on different angular surfaces of the work body.

In accordance with the invention there is provided a shaping instrument for the preparation of adjacent surface of a work body including: a contoured body having engagement elements on an active surface to work a passive surface of a work body; a transfer mechanism connectable to a rotatable drive and engageable with the contoured body; wherein the transfer mechanism converts the rotational movement of the rotatable drive to a combination of at least one small local movements of one or more parts of the contoured body, with or without regional rotational movements that when employed, circumferentially advance the engagement elements on the passive surface of the work body; wherein the shaping instrument is able to work the passive surface of the work body by the combination of at least one regional movement, advancing slowly in a clockwise direction.

Preferably the small local movements are rotational oscillating movements around the drive axis of the rotatable drive.

In another form the small local movements are rotational movements around a respective radial axis offset to the drive axis of the rotatable drive.

Preferably the contoured body is formed of a plurality of parts which each are simultaneously driven to provide a plurality of small local movements to work the passive surface of the work body.

The plurality of small local movements of the plurality of parts of the contoured body to work the passive surface of the work body can be rotational oscillating movements around a common axis.

However, the plurality of small local movements of the plurality of parts of the contoured body to work the passive surface of the work body can be rotational movements around a plurality of respective radial axes offset to the drive axis of the rotatable drive.

Preferably the plurality of respective radial axes offset to the drive axis of the rotatable drive extend from a Single Point Centre of Rotation (SPCOR).

The engagement elements of the contoured body are one or more of the type that:
Manipulate
sense
Deform
Cut
Shave
Pierce
Gouge
Abrade
Resect

In a further aspect of the invention there is described a bone oscillating instrument adapted to shaping or contouring of underlying of overlaying bone with or without a secondary use of structural support and/or use of the instrument as a definitive prosthesis comprising:
a) a primary driver
b) as oscillating instrument
c) a gearing mechanism intermediate the primary rotating driver and the oscillating instrument
d) wherein the gearing mechanism is adapted to covert rotation movement to a rapid oscillation movement so that the oscillating instrument contours, shapes and cuts underlying bone.

The novelty of one aspect of this invention lies with the mechanism of action of the instrument and the additional use of the instrument as a definitive implantable structural element or prosthesis.

The present invention has been designed to mitigate against soft tissue injury when contouring or sculpting a bone by substituting large distance bone reamer movement for fast, small oscillatory movements. The reduction of large-scale movements also reduces large distance translations of sharp surfaces that can inadvertently injure adjacent non-bone structures.

The small range excursions also mitigate against the sudden transfer of large forces to the wrist and hand of the surgeon/device operator.

The present invention can incorporate a non-cutting region that prevents error in depth of reaming and assists in achieving target direction of reaming during the bone preparation.

Preferably the oscillating instrument/prosthesis comprises a contouring cutting shell. The cutting shell can comprise an inner and outer surface. The active surface exhibits a number of elements for piercing, gouging, abrading or resecting bone.

The wall of the instrument exhibits repeating inner structural voids that permit the passage of reamed bone from the outer to the inner shell surface. The voids can be generally circular in shape or be designed with freedom to allow bone to egress from the shaped surface to the inner aspect of the contour body.

Preferably the gearing mechanism comprises: an oscillation plate (first transfer mechanism), and a second transfer mechanism

In one embodiment, converting rotation movement to a rapid oscillation movement which can be by rotation around an axis. In an alternative embodiment the movement can be translationally aligned with an axis. The resultant movement can be a complex combination of rotation and translationally aligned axial movement.

Preferably the gearing mechanism intermediate the primary rotating driver and the oscillating instrument/prosthesis is quick releasable.

A gearing mechanism that coverts rotation movement to a rapid oscillation movement which can be rotation around an axis, or where the movement can be translationally aligned with an axis or a complex combination of rotation and translationally aligned axial movement.

A bone oscillating instrument/prosthesis designed to use the gearing mechanism oscillation movements to a shaping or contouring of the underlying of overlying bone with or without a secondary use of structural support and/or use of the instrument as a definitive prosthesis.

A gearing mechanism that converts the uni-directional rotational movement of a drill driver, to a rapid oscillating movement;
a. Where contouring and shaping is required, the preferred movement is rapid rotational oscillation;
b. Where impacting the instrument as a structural support or as a definitive prosthesis, the preferred movement is rapid oscillating/impaction movement.

The gearing mechanism is the interface between the primary rotating driver and the oscillating instrument/prosthesis. The gearing mechanism will exhibit a quick coupling capability between both instrument and driver.

The details of the gearing system options are best described in conjunction with illustrations (see below). In general, there are two components of the gearing mechanism. The components are (1) the oscillation plate (first transfer mechanism) and (2) the second transfer mechanism. These two components will vary in design as the movement mechanism and the oscillating instrument are varied.

A bone oscillating instrument/prosthesis, comprising an inner and outer surface. The active surface exhibits a number of elements for piercing, gouging, abrading or resecting bone.

The wall of the instrument exhibits repeating inner structural voids that permit the passage of reamed bone from the outer to the inner shell surface.

The internal architecture of the instrument can comprise purposefully designed structural elements used to collect and disperse autogenous bone graft generated from the bone reaming or for interfacing with other prosthetic components. The internal architecture of the instrument can also allow for impaction of autogenous bone to the previously shaped work body surface.

The shape of the reaming instrument is variable so as to facilitate site specific shaping and contouring of bone. A custom shape of the instrument will allow for site specific structural support and/or site-specific instrument/prosthesis implantation. The instrument shape for surgery on the inner acetabulum will be largely hemispherical and will be different from the hemispherical instrument used to shape the outer surface of the femoral head during re-surfacing procedures.

The instrument body can have the structural design of a triangulated mesh to allow enhanced structural integrity of the instrument.

The instrument will generally comprising an active cutting zone and a non-cutting zone. The purpose of the non-cutting zone of the instrument allows a regulation of direction and depth of bone contouring/shaping during bone preparation.

The primary function of the instrument is to shape/contour bone by rapid oscillating movements.

A second purpose of the instrument can be to act as structural support to the bone when bone deficiency is evident or where there is a requirement to stabilize one or more bones, with or without additional internal fixation. Indications for such use would include revision knee or hip surgery where volume bone voids of the femur, acetabulum or tibia are bridged by the autogenous bone grafted site-specific instrument. The design of the instrument can facilitate the entrapment of autogenous bone graft into the wall structure of the instrument. This feature can enhance bone ingrowth to the instrument acting as a structural support.

A third purpose of the instrument is to remain in situ, with or without additional internal fixation, and used as an implantable prosthetic component. The instrument/prosthesis will typically contain compartmentalized autogenous host bone generated at the time of rapid oscillating movements of the instrument.

The bone reaming shell can be made from a range of material including, but not limited to stainless steel, titanium, chrome cobalt alloy, a plastic polymer or photosensitive resin or a combination of materials metal/plastic combination. The manufacturing process can include stamping, 3d printing, injection molding or conventional milling of materials.

Components of the invention can be single use or multiple use.

It can be seen that the driving of the movements of the combination of local and regional are achieved (the driving mechanism or transfer mechanism) is fundamentally different.

The first novel difference is that the present art is able to shape both concave and convex surfaces. This is a “material” difference between the inventions and the prior art.

The second point of difference is that the drive mechanism (transfer mechanism) of the present invention is an indirect “semi-linked”, “clutch” drive mechanism that allows a variable amount of independent rotation between first and second transfer mechanism. The present art rotation movement in the transfer mechanism is fundamental to an understanding of the transfer of poser and the generation of local movement

The third point of difference (and advantage) of the present art is that is that the ratio of local and regional movement can be regulated with more or less exertion by the user through the instrument Increasing user pressure exertion on the instrument will increase the engagement between the first and second transfer mechanism (akin to a “clutch mechanism”) which increases the relative local movement of the invention.

When seen from a different perspective, a decrease in pressure applied by the user though the invention decreases the engagement between first and second transfer mechanisms and increases the local movement of the invention when compared with regional movement. The points made to the regulation of local and regional movements in the present art are equally applicable to both concave and convex reamers.

The 4th point of difference and advantage of the invention is the use of modularity in the contour body (and associated cutting elements) to regulate the depth of resection of the shaped body. The “polar” reamer described can only deepen the resection on both concave or convex surfaces to the distance that the cutting elements protrude from the non-cutting contour body. This adds a significant safety factor to reamer use during orthopedic surgery.

The 5th point of difference and advantage of the invention is the present invention uses modularity in the second transfer mechanism and in the engagement elements to allow the collection and impaction of bone to the shaped surface. A quality surface finish with enhanced structural integrity (post bone impaction) is fundamental to end goals of a finished product during orthopedic surgery.

### BRIEF DESCRIPTION OF THE DRAWINGS

Not withstanding any other forms which can fall within the scope of the present invention, preferred embodiments of the invention will now be described, by way of example only, with reference to the accompanying drawings in which:

FIG. 1 shows a diagrammatic cross-sectional view of the structural elements of a shaping instrument in accordance with an embodiment of the invention

FIG. 2 shows a diagrammatic functional perspective view of the shaping instrument of FIG. 1 showing the relative rotation of the drive shaft and base swash plate providing the applied first transfer mechanism;

FIG. 3 shows a diagrammatic functional perspective view of the shaping instrument of FIG. 1 showing the relative rotation of the drive shaft and base swash plate providing the applied second transfer mechanism, where the second transfer mechanism is a slave to the movement of the first transfer mechanism is all directions except rotational movement;

FIG. 4 is a diagrammatical perspective view of the driven convex curved body by the first and second transfer means of FIGS. 1, 2 and 3 to effect movement of the extending engagement elements to contour the passive surface of the work body;

FIGS. 5 and 6 are perspective diagrammatic views of the local circular movement (T) of an active engagement element 25 around a radial axis (D) between the active engagement elements and the Single Point Centre of Rotation (C).

FIGS. 7 and 8 are underneath diagrammatic sectional perspective views of an active engagement element placed on contoured body with the underside internal cavity of the work body shown where the movement of the active engagement element shapes and contours the passive surface of the work body.

FIGS. 9 and 10 are diagrammatic perspective views of a regional and local movement patterns resulting from the first and second mechanism where the first transfer mechanism can provide a large regional movement (S) of the driven convex curved body but in addition to local movement pattern (T), the active engagement element 25 also circumferentially rotate around an axis central to the contoured body (A). The summate effect is “simultaneous” regional (S) and local movement patterns (T) of the active engagement elements.

FIG. 11 is a sectional view of a combination of drive shaft and first transfer mechanism being a rotating plate set at angle to the drive shaft

FIG. 12 is a diagrammatic perspective view of a second transfer mechanism in which the second transfer mechanism is a rotating bearing where the rotation of the inner bearing is independent of the rotation of the outer bearing to effect the movement of the second transfer mechanism to be a slave to all movements of the first transfer mechanism with the exception of rotation movement

FIG. 13 is a perspective view of a preferred embodiment of a contoured body showing active engagement (cutting) elements and structural voids to allow transfer of material from the work body through to the inner chamber of the contouring body. Note the cutting elements have a choice of shape, number and direction.

FIGS. 14 and 15 are connected and exploded perspective views of an assembly of parts comprising a drive with first transfer mechanism, a second transfer mechanism and a coupler to a contour body;

FIGS. 16 to 20 are diagrammatic views of other embodiments of contouring body with spiral edges, contouring bodies with a plurality of pyramid cutting elements arranged in a geodesic pattern with and without polar “non-reaming” surface;

FIGS. 21 and 22 are diagrammatic cross-sectional views of a comparison of a concave shaping instrument such as in FIGS. 1 to 20 for working on an inside cavity of a work body and the concepts that can be similarly applied to a convex shaping instrument for working on an outside proud surface of a convex work body;

FIGS. 23, 24 and 25 are diagrammatic cross sectional views of a convex shaping instrument for working on an outside proud surface of a convex work body;

FIG. 26 is a diagrammatic view of a non-symmetrical convex shaping instrument as a further embodiment of the invention;

FIG. 27 is a diagrammatic view of the shaping by use of an adjustable depth standard reamer to contour a standard polar depth region;

FIG. 28 is a diagrammatic view of use of a further concave shaping instrument such as shown in FIG. 18 as an embodiment of the invention without a polar region but with shapings and voids to effect different localised contouring in the work body away from the polar region;

FIGS. 29, 30, 31, 32, 33, and 34 are axial and perspective views of the invention for use in one embodiment on a proximal tibial with bone defect, with placement of a contour body to shape and support the bone void proximal tibia demonstrating the application of the instrument as a structural element to manage a proximal tibial bone defect when performing TKA.

FIG. 35 is a diagrammatic view of a direct drive of the first and second transfer mechanisms by use of interface therebetween and use of reduction gears to limit speed relative to drive of the rotatable drive.

### DETAILED DESCRIPTION

### General Structure of Regional Movement

The instrument will generally comprising an active cutting zone and a non-cutting zone. The purpose of the non-cutting zone of the instrument allows a regulation of direction and depth of bone contouring/shaping during bone preparation.

The angle between first transfer mechanism 30 of the base swash plate 31 and drive shaft 32 is important. Smaller angles lead to smaller local movements of engagement elements. A 0° angle (ie not a swash plate) will result in only “all regional movement (S)” (or all coaxial movement) and no “local movement pattern (T)” by the contoured body engagement elements.

As shown the design of the shape of the contoured body can be varied. In a preferred embodiment, the contoured body is hemispherical and contains engagement or cutting elements and void areas for egress of working product (swarf). In a preferred embodiment, the polar area of the contour body 20 is devoid of cutting elements 25 so that (accidental) unrestricted advancement of the contoured body into the work body is restricted. A plurality of pyramid cutting elements can be arranged in a geodesic pattern that allows enhanced structural integrity of the contour body and enables the contoured body to be used as a stabilising product or a prosthesis. Small voids 26 can be located between the engagement elements 25.

In order to further enhance capability of working on a work body the shaping instrument can be used in different forms and in different combinations.

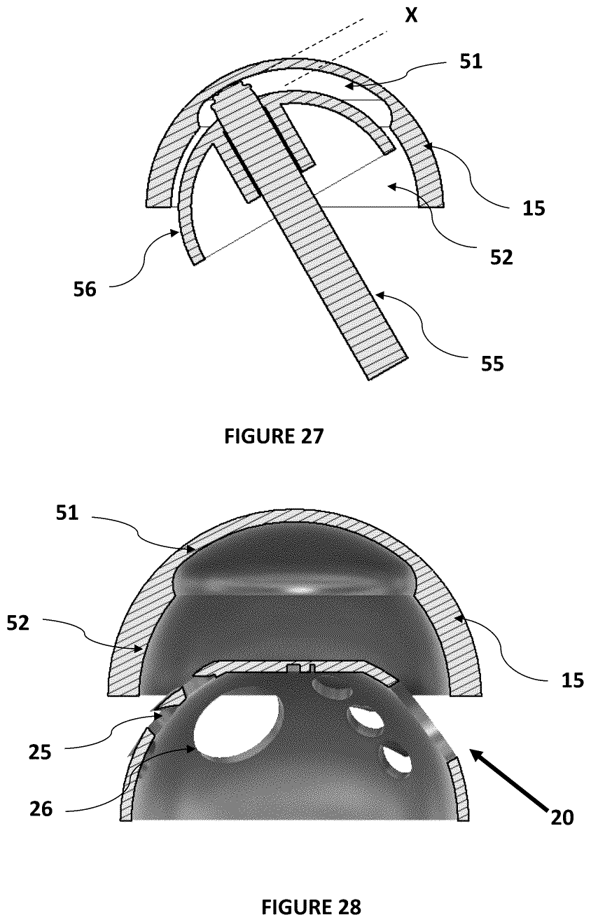

In the embodiment of FIGS. 27 and 28, the shaping of a work body 15 can be considered as a one or two stage shaping process. In this preferred process, a two-step process is applied at different depths.

FIG. 27 demonstrates a non-cutting convex acetabular shell engaging with a concave work body (acetabulum). A standard reamer 55 acting as a depth “router” now protrudes from the acetabular shell 56, into the polar region of the work body 15 by a defined distance X. The “pilot hole” is thereby formed. Rotating the acetabular guide within the acetabulum, will engage the router to the known depth and in a direction determined by the surgeon. Thereby there is a polar contouring to a known depth and known direction as the “first stage” of the two-stage acetabular shaping process.

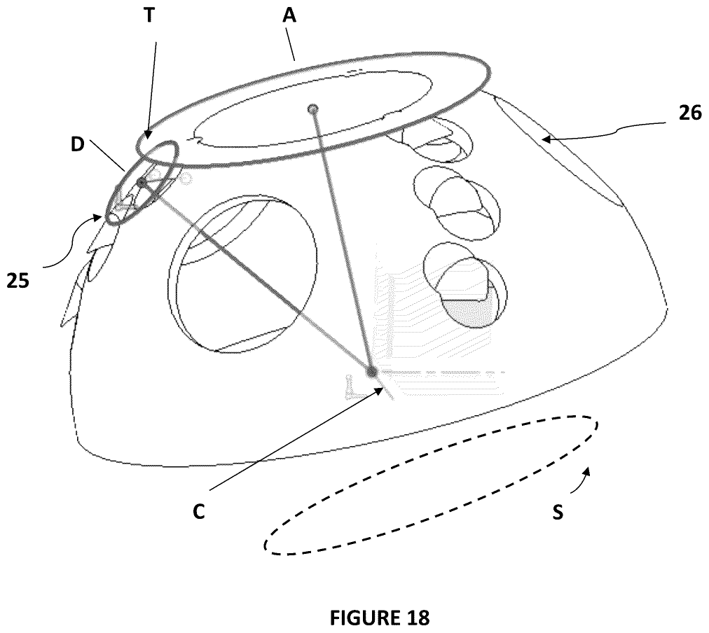

FIG. 28 demonstrate the resulting contouring of the first stage at the polar region 51 at the known depth X and known direction by the pilot hole “router”. In the second stage, once this pilot hole with known depth is prepared, the previously described acetabular reamer will advance until the “non-cutting” polar aspect of the reamer reaches the prepared floor. In this way, accidental excessive depth resection and wayward directional changes found in preparation of the acetabulum with traditional methodology will be effectively mitigated. Use of the work body 20 of the shaping instrument 11 without a polar cutting section, similar to the embodiment of FIG. 18 will result in controlled localized contouring around the engagement elements 25 in only the non-polar region 52.

A two-stage process, as described, for shaping the acetabulum during hip arthroplasty ensures a better safety and quality profile of acetabular preparation.

In one embodiment the instrument has a cutting zone and a non-cutting zone. The purpose of the non-cutting zone of the instrument allows for the regulation of direction and depth of bone contouring/shaping during bone preparation.

General Structure of combination of regional and local movement

This combination can be a combination of local and regional movement provided by a rotational drive means and one or more transfer means.

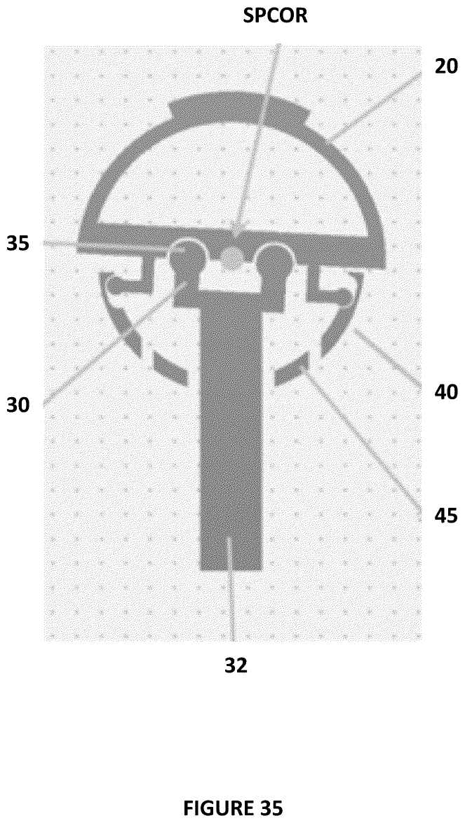

Referring to FIG. 35, this diagram has the features of a rotatable drive, a first and second transfer mechanisms and a contour body 20 that is customized to shape the passive surface. The rotatable drive 32 rotates around a longitudinal axis to a first transfer mechanism 30 that is set obliquely to the longitudinal axis of the rotatable drive. This oblique plate at the top of the first transfer body captures the contour body 20 and drives all movement with the exception of rotation of the contour body around the longitudinal axis of the rotatable drive.

If friction is applied to the contour body 20 and absent the second transfer mechanism 40 then the contour body would wobble around its single point centre of rotation (SPCOR) which is coincident with the central axis of the rotatable drive. This interface between the rotatable drive, the first transfer body and the contour body sets up local movements of the cutting elements of the contour body. In effect these are circular movements of the cutting elements on the surface of the contour body which move around a radial axis between the cutting elements and the single point centre of rotation.

In this embodiment the rotatable drive also drives regional movement of the contour body. In this embodiment the rotatable drive interfaces with reduction gears 45 that reduce the regional movement of the contour body 20.

In the diagram shown the interface 35 between the second transfer mechanism 40 and the contour body 20 captures and drives regional movement of the contour body around the longitudinal axis of the rotatable drive 32. The region movement also trots around the SPCOR as this is co-incident with the longitudinal axis of the rotatable drive.

The interface between second transfer mechanism 40 and the contour body 20 does not interfere or change the movement generated by the first transfer mechanism 30 however the regional movement of the contour body 20 is a slave to the regional movement of the second transfer body 40. In effect the combination of the two movements generated by the first and second transfer bodies 30, 40 to the contour body 20 actions a combination of small, fast, rotary movements of the cutting elements around a radial axis between the cutting elements and the single point centre of rotation (SPCOR) and also the second movement of regional movement which may be geared by the reduction gears 45 to be slower than the rotation speed of the rotatable drive 32.

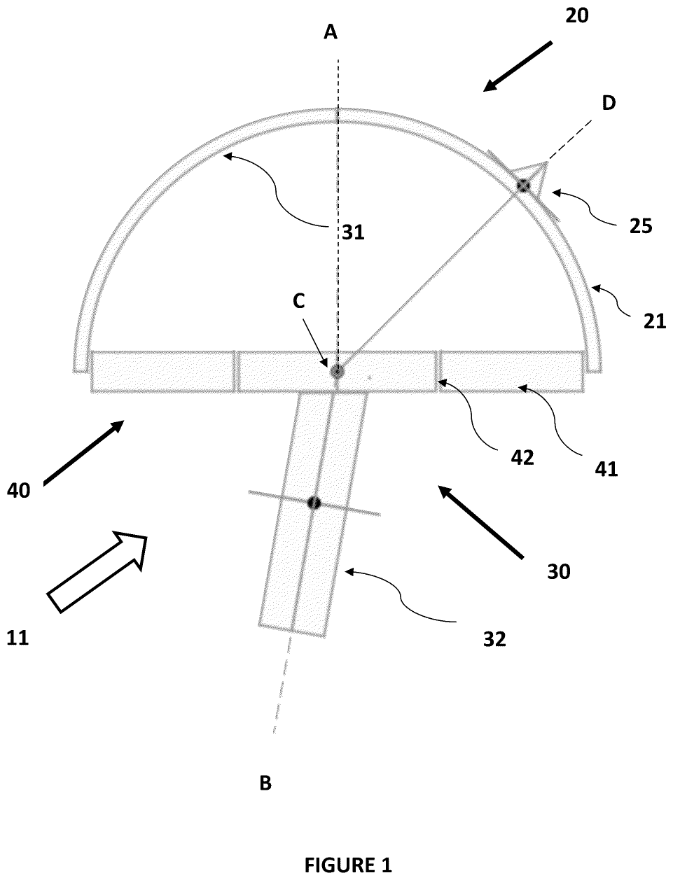

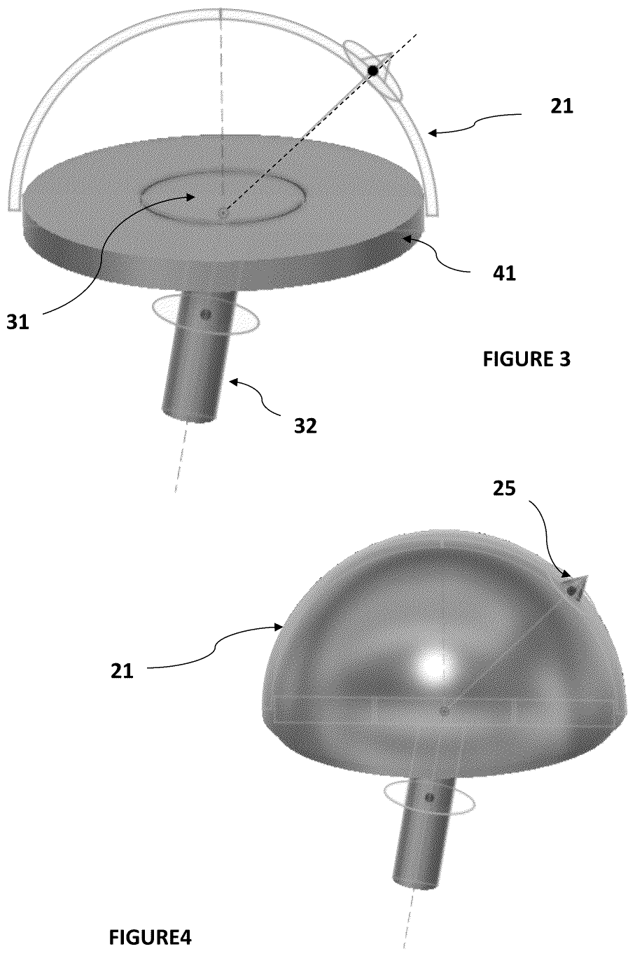

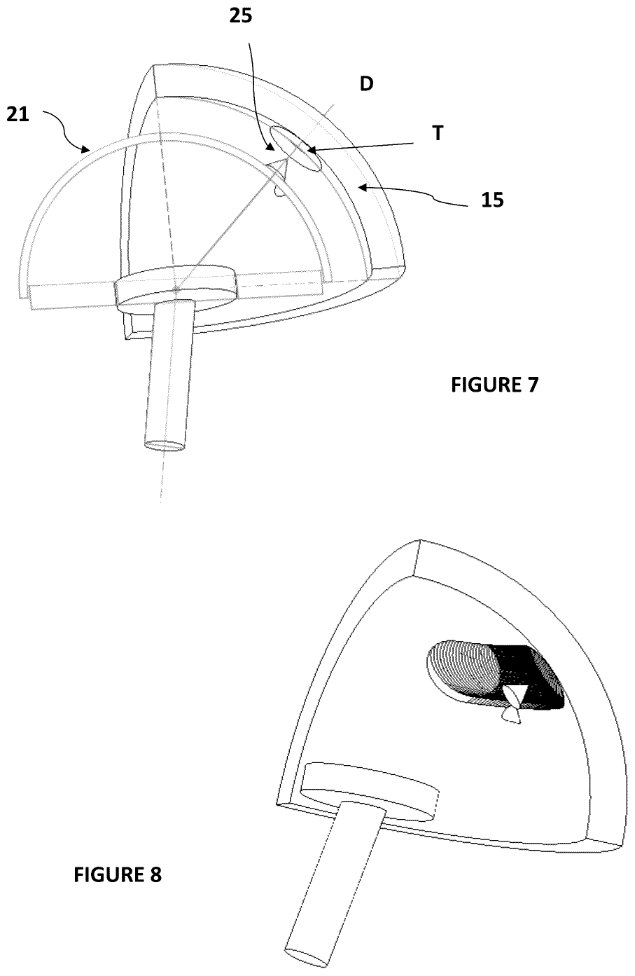

Referring to the drawings and particularly FIGS. 1 to 10, there is provided a shaping instrument 11 for the preparation of a concave inner surface of a work body that operates similarly but uses friction. The local movement pattern prevails when friction is applied to the contoured/engagement elements so driving the independent rotation between first and second transfer mechanisms.

The structural elements of the shaping instrument 11 include a contoured convex curved body 20, a first transfer mechanism 30 and a second transfer mechanism 40. Each of the structural elements can be “special purposed” to perform specific functional requirements.

The contoured convex curved body 20 of the shaping instrument 11 is in the shape of a hemisphere. Outwardly protruding engagement elements 25 extend from an outer surface 21 of the convex curved body. These protruding elements 25 are to work an inner surface of a work body.

The shaping instrument 11 further includes a two-piece transfer mechanism having a first transfer mechanism 30, and a second transfer mechanism 40 connectable to a rotatable drive for effecting movement of the contoured body 20.

The first transfer mechanism 30 comprises a base swash plate 31 formed by an angular offset of a base swash plate 31 to an angled elongated tubular drive shaft 32. This effects a Single Point Centre Of Rotation (SPCOR) at (C) which is the centre of rotation around which the transfer mechanism rotates and is typically co-incident with the longitudinal axis (B) of the drive shaft 32.

In operation, the rotatable driveshaft 32 of the shaping instrument 11 connects to a driver such as a commercial power drill. The drive shaft typically rotates quickly so as to enable rapid oscillation movements through the elements of the shaping instrument 11. The drive shaft 32 forms part of the first transfer mechanism 30 and by connection to the base swash plate 31 at an angular offset to the longitudinal axis of rotation of the drive shaft effects the first transfer means.

The single point centre of rotation (SPCOR) of the first transfer mechanism 30 is typically co-incident with the longitudinal axis of the drive shaft 32. Design modifications allow for a translational offset between the centre of axis of the first transfer mechanism and the longitudinal axis of the drive shaft leading to an offset rotation of the first transfer mechanism with reference to the longitudinal axis of the drive shaft.

Rapid rotation of the drive shaft 32 leads to a wobble or precession of the swash plate 31 of the first transfer mechanism 30, in relation to the longitudinal axis (B) of the drive shaft 32. Larger angles between drive shaft 32 and swash plate 31 causes greater precession or wobble of the plate as it rotates.

The shape and size of the first transfer mechanism 30 varies with functional requirements. The angle between drive shaft 32 and the base swash plate 31 of the first mechanism varies between 0-90°, but typically is in a range 1-20°, as this allows for rapid directional change (oscillations) between the cutting engagement elements 25 of the contour body with reference to the longitudinal rotation of the drive shaft.

The drive shaft is typically rotating quickly at “drill speed” of about 1500-2500 RPM enabling rapid local oscillation movements of the engaging protruding elements 25 over small distances.

The second transfer mechanism 40 is coupled to the first transfer mechanism 30 and is a slave to all movements of the first transfer mechanism with the exception of rotation movement. The second transfer mechanism 40 is a coaxial annular plate 41 joined by bearing coupler 42 so it is able to independently rotate around its own longitudinal axis of rotation (A) as distinct to the drive angle of the longitudinal axis (B) of the drive shaft 32.

The convex curved body 20 has one or more attached outwardly protruding engagement elements 25 which are coupled to the second transfer mechanism 40 by the convex curved body 20 attached to the diametrically outer edges of the coaxial annular plate 41 of the second transfer mechanism 40. The contoured body movement is a slave to the first transfer mechanism movement in all directions with the exception of rotation due to the rotation joint of the contact bearing 42 between first and second transfer mechanisms. Therefore the outwardly protruding engagement elements 25 precess around the axis of rotation D extending from the Single Point Centre Of Rotation (SPCOR) (C) and the axis of the outwardly protruding engagement elements 25.

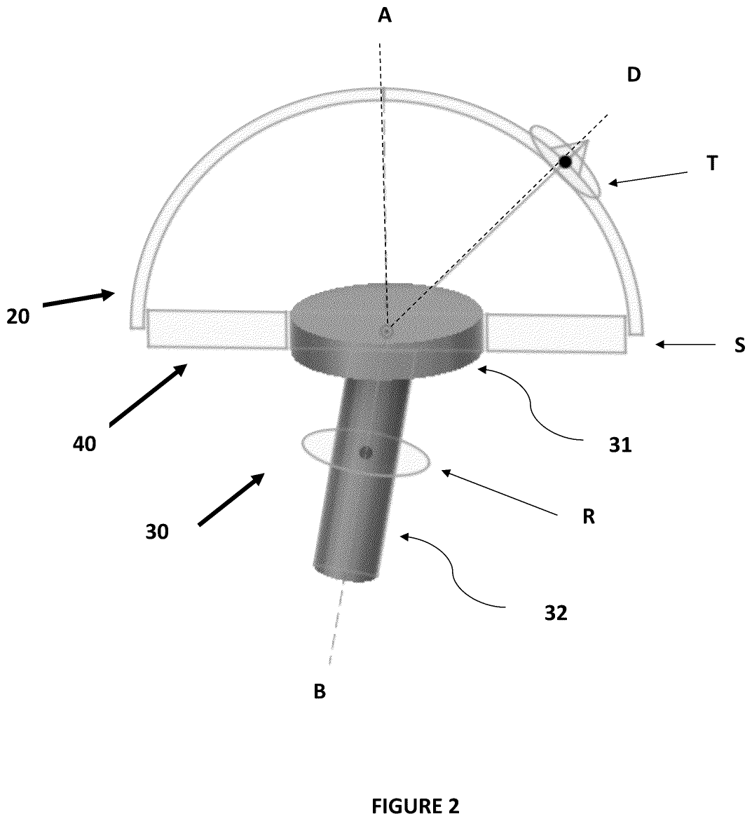

As shown in FIGS. 2, 3 and 4, the result of rotation R of the first transfer mechanism 30 around axis B of the and the slave movement of the second transfer mechanism 40 to the first transfer mechanism 30 with the exception of rotation movement results in the precessing movement T of the outwardly protruding engagement elements 25 around axis (D) on the surface of the convex curved body 20.

Looking at the complex movement patterns of the engaging elements 25 it can be seen that the transfer mechanisms convert the rotational movement of the rotatable drive 31 to at least one small local movement (T) of one or more parts 25 of the contoured body 20, wherein the shaping instrument 11 is able to work the work body surface by the at least one small local movement.

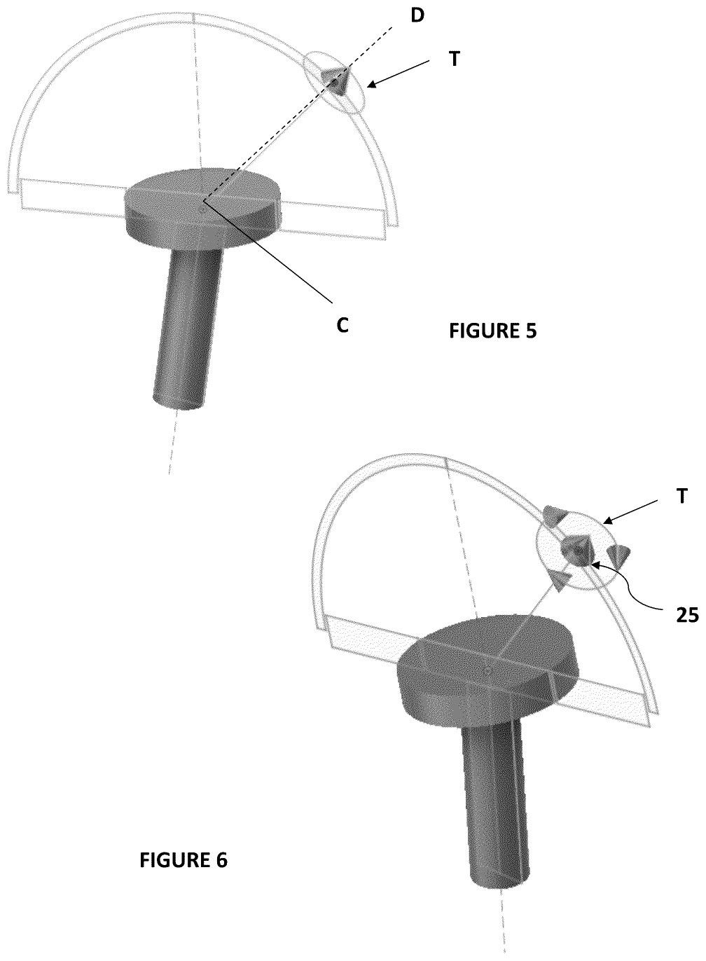

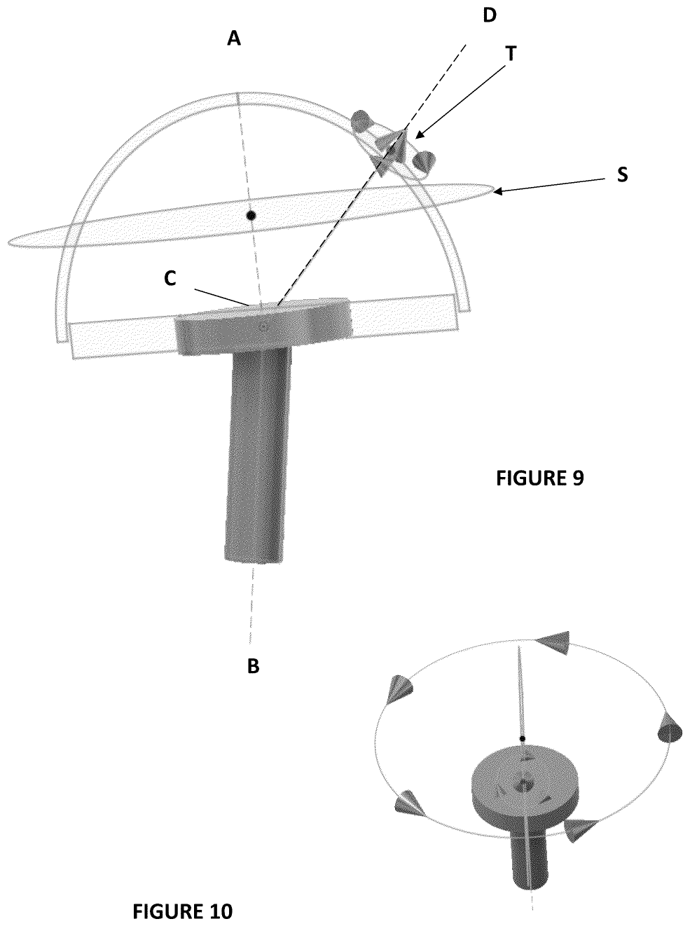

Referring to FIGS. 5 to 10, there is demonstrated that rotational movement of the drive shaft 32 does not usually provide total rotational movement of the whole work body surface of the contoured convex curved body 20 such as movement S in FIG. 9, but provides a local movement pattern (T) of the of the outwardly protruding engagement elements 25 around its local axis D. This local movement pattern (T) prevails when friction is applied to the contoured/engagement elements 25 so driving the independent rotation between first and second transfer mechanisms 30, 40.

The local movement pattern (T) is a circular pattern as shown in FIG. 6 around a radial axis (D) between the engagement element 25 and the SPCOR (C). Each engagement element 25 produces its own local shaping pattern (T) in the work body.

The size of the local shaping (T) is a function of the length of the radial axis (D) and the angle subtended between the first transfer plate 31 and the drive shaft 32 and therefore between axis (A) and axis (B).

As friction to the contour body is reduced, the movement pattern of the contour body 20 (and engagement elements 25) changes. When friction applied by the work body to the contour body is negligible, the second transfer mechanism 40 rotationally moves with the first transfer mechanism 30. Therefore, the local movement pattern (T) transitions to regional circumferential movements (S) of the engagement elements as shown in FIG. 9. In practice, the reduced friction environment occurs after the work body has been shaped by the cutting elements of the contour body.

The reduced friction environment leads to a regional rotation of the cutting elements to meet “un-shaped” work body when friction again increases and local movement patterns (T) again prevail. This cycle is continuously repeated until there is no further work body to shape by the chosen contour body. The shaping on the work body occurs with a combination of (cycling) local and regional movement patterns of the engaging elements 25. The combination of local and regional movements results in a sweeping circumferential shaping of the work body around the longitudinal axis of the drive shaft. A plurality of engagement elements 25 result in plurality of shaping sweeps.

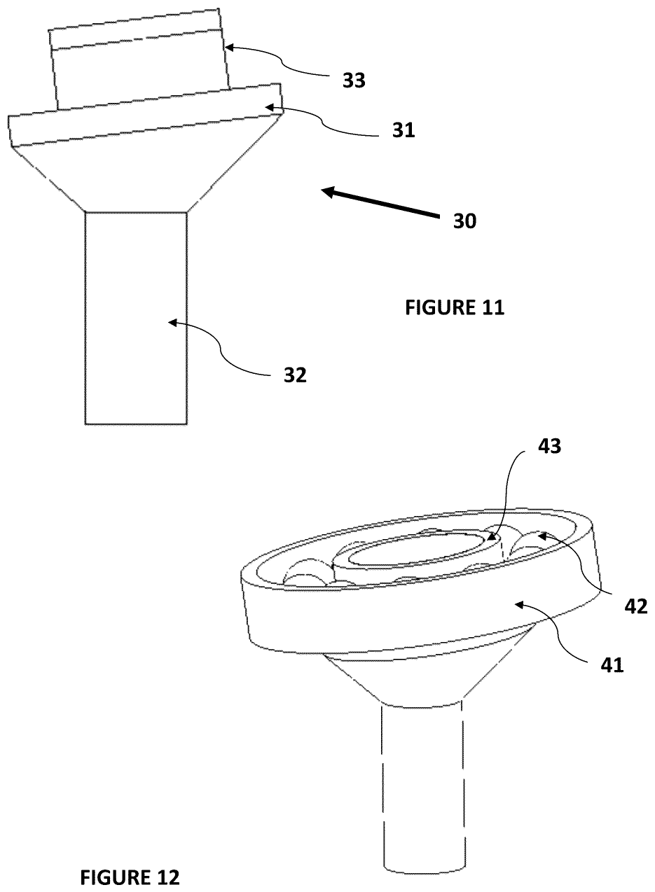

Referring to FIGS. 11 to 15, the coupling of the drive shaft 32 and the base swash plate 31 of transfer mechanism 30 is typically a rigid joint. It is at an angle to each other so that rotation of the drive shaft around its axis will effect rotation of the base swash plate at its offset angle. This provides a swash plate effect. From the top of the base swash plate 31 extends a centering connector extension 33 for mounting the second transfer mechanism 40 in the form of a bearing coupler. Clearly such mounting could be to perimeter of the centering connector extension 31.

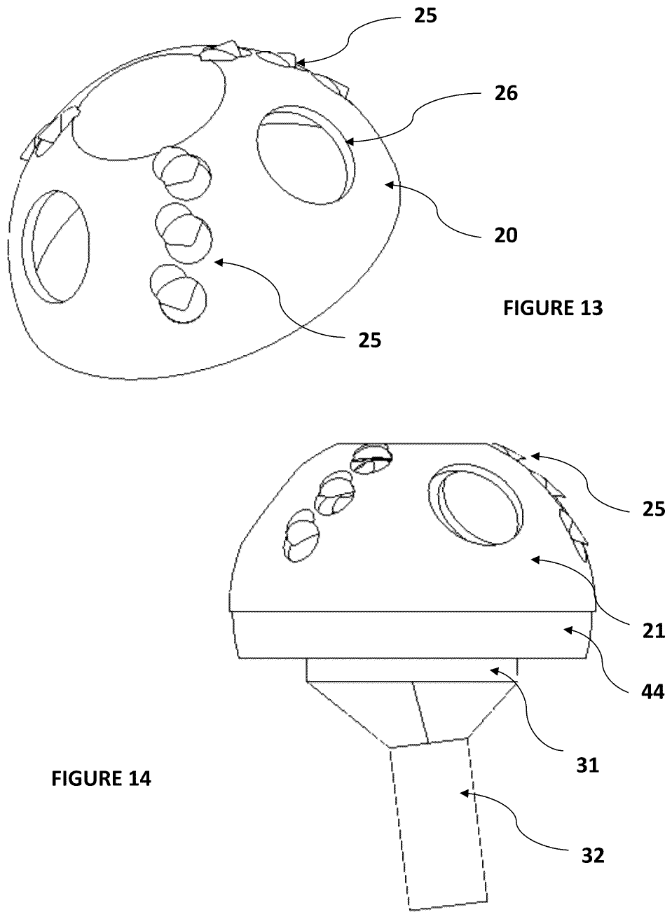

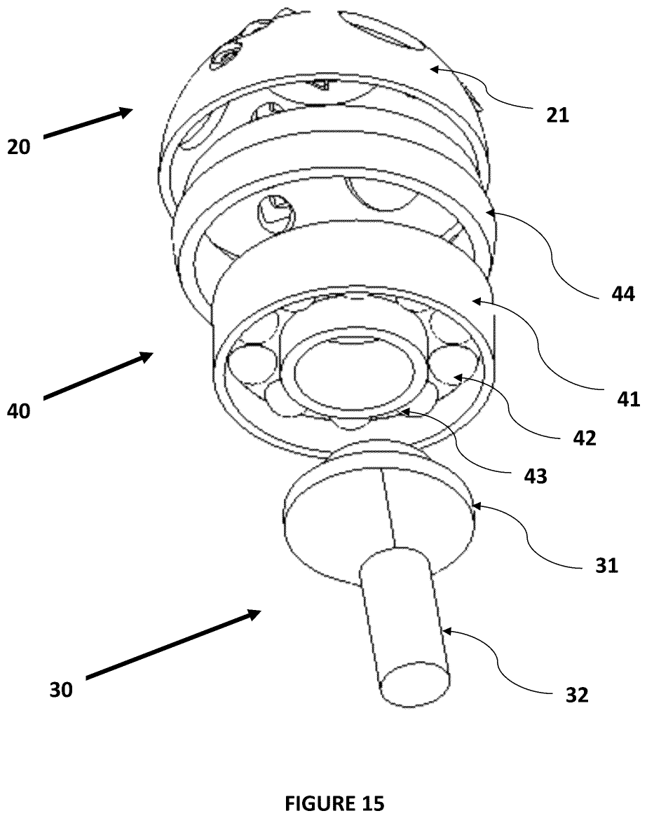

FIG. 15 shows a disassembled construct and FIG. 14, a partly assembled construct of a drive shaft 32, first 30 and second 40 transfer mechanisms, coupled to a contoured body 20. The fully assembled components and a exploded view demonstrate the relationships of the components to each other.

The angle between first transfer mechanism 30 of the base swash plate 31 and drive shaft 32 is important. Smaller angles lead to smaller local movements of engagement elements. A 0° angle (ie not a swash plate) will result in only “all regional movement (S)” (or all coaxial movement) and no “local movement pattern (T)” by the contoured body engagement elements.

A large angle between first transfer mechanism 30 of the base swash plate 31 and drive shaft 32 will result in excessive precession of the first transfer mechanism 30. Typically, an angle between 1°-20° will be applied to this rigid couple between drive shaft and first transfer mechanism. Higher angles (20-90°) between first transfer mechanism and drive shaft are used with slower drive shaft RPM. FIG. 11 shows a first transfer mechanism at about 10° to the longitudinal axis of the drive shaft.

The purpose of second transfer mechanism 40 is to permit independent rotation movement between first transfer mechanism 30 and the attached contouring body 20 when friction is applied to the contour body (by application to the work body). FIG. 12 shows a bearing coupling formed by bearings 42 mounted between outer annular ring 41 and inner ring 43 that can be mounted on the centering connector extension 33 and permit independent rotation between inner part 31 of the first transfer mechanism 30 and outer annular part 41 of the second transfer mechanism 40. As shown by FIG. 15 there is shown the coupling of the bearings within the annular ring 41 to a mounting ring 44 that frictionally fits to an inner surface 42 of the second transfer mechanism 40 to the first transfer mechanism 30 and drive shaft 32.

The coupling between first transfer mechanism 30 and the is by the second transfer mechanism 40 acting as a rotation joint. The second transfer mechanism 40 moves with first transfer mechanism 30 in all directions with exception of rotation movement. As the first transfer mechanism has precession, so does the second transfer mechanism. Note that the shape of the first transfer mechanism assists in the direction of movement of the second transfer mechanism in all directions except for rotation.

Both first and second transfer mechanism have a single point centre of rotation C which is typically aligned with the longitudinal axis of the drive shaft. This design feature directs a symmetrical shaping of the work body around the SPCOR.

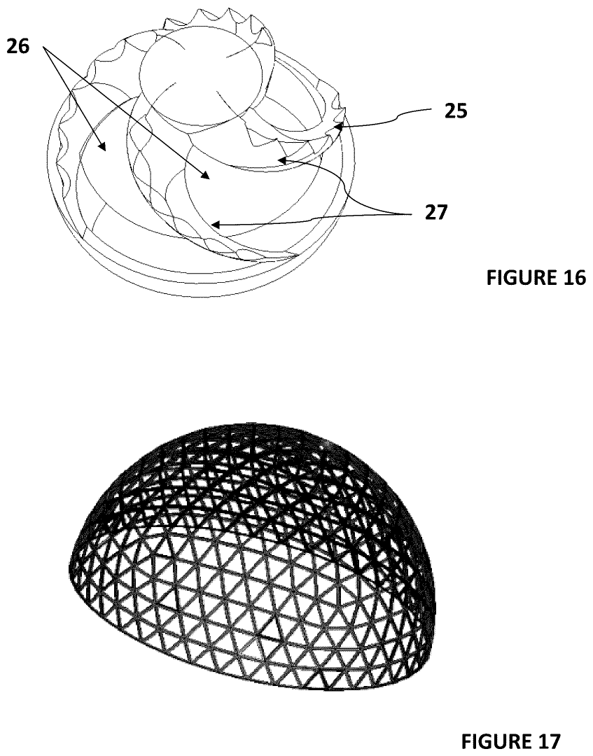

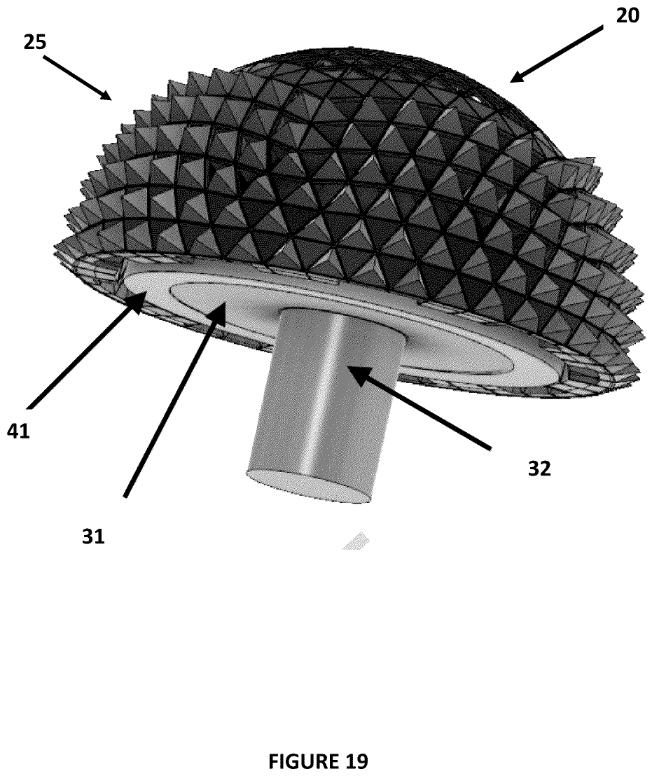

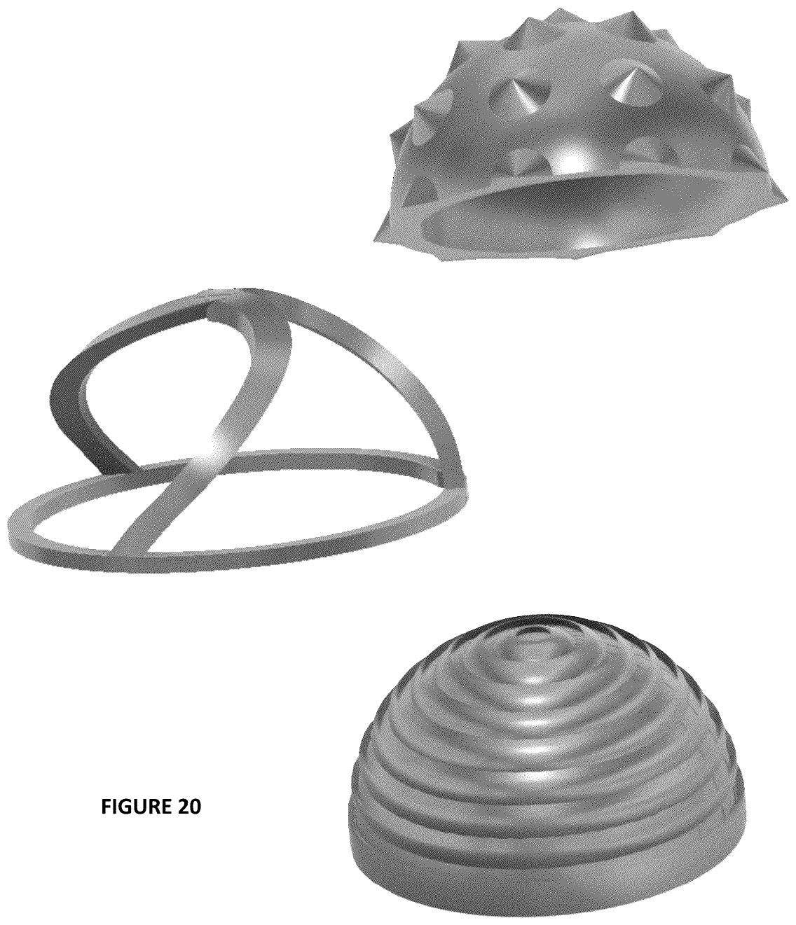

Referring to FIGS. 16 to 20, the purpose of the contoured body 20 is to shape the work body by a combination of local T and regional movements S of the engagement elements 25 attached to the contour body 20. The contour body 20 is generally hemi-spherical or frusto-spherical in design. Apart from an arrangement of similar sized or differently sized outwardly protruding engagement elements 25 on an outer surface 21 of the convex curved body 20 there can be one or more voids 26 to ensure non-working at that location of the work body. However, those voids could have circumferential inwardly facing cutting edges that provide a smoothing of the work body at that location.

Referring to FIGS. 13 and 16 to 20 there is shown a plurality of differing outer bodies 20 having differing configurations of outwardly protruding engagement elements 25 that can make use of the novel regional movement (S)” and “local movement pattern (T)” by the contoured body engagement elements 25 to effect required action on the inner surface of the work body at the required localized site. A contoured body 20 could be a component of a definitive prosthesis (B) used in joint arthroplasty. Unusual individual and continuous cutting elements can form part (or all) of the contour body. Unusual contour body profiles can effectively take advantage of the innovative (combination) movement pattern of the invention.

As shown the design of the shape of the contoured body can be varied. In a preferred embodiment, the contoured body is hemispherical and contains engagement or cutting elements and void areas for egress of working product (swarf). In a preferred embodiment, the polar area of the contour body 20 is devoid of cutting elements 25 so that (accidental) unrestricted advancement of the contoured body into the work body is restricted. A plurality of pyramid cutting elements can be arranged in a geodesic pattern that allows enhanced structural integrity of the contour body and enables the contoured body to be used as a stabilizing product or a prosthesis. Small voids 26 can be located between the engagement elements 25.

An example of the contour body containing voids 26 such as shown in FIG. 13 and that enable the egress of work material to move from the surface of the work body to an area within the contoured body. Spherical voids are designed for this purpose of egress.

FIG. 16 demonstrates an alternative embodiment of contour body where the design is characterized by a series of spiral arms 27 that remain contoured to the general hemispherical or frusto-spherical shape. The externally directed cutting elements 25 are on the leading edge of each arm and the “voids” 26 are the separation between each of the spiral arms 27. Note that in this preferred embodiment, the cutting elements have a shape which is pointed at the presenting (leading) edge, and generally directed forward and up. The concave shape of the spiral arms facilitates a scooping and containment of the work body swarf.

In a preferred embodiment, the engagement elements are variably designed to be facing forward/up and forward/down. No two elements are oriented in the same direction. Note that the variation in direction of the engagement elements means that at any one time, only one of the many engagement elements of the contour body will be effectively engaged at any particular time. This is important as it means that the effective “working contact” between contour body and work body is small and therefore the drive mechanism is able to work at low torque and high rotation speeds.

The orientation of the engagement elements with reference to the longitudinal axis of the rotatable drive, ranges from 0° (facing directly up or directly down), to facing directly in a clockwise direction, (+90°) or in a counterclockwise direction (−90°). There can be variation in direction of cutting elements (A), with bias in a clockwise direction.

The cutting elements are “purposefully engaged” when they are moving in the direction of the sharp leading edge of the cutting element. As the primary cutting movement is circular (local movements), then the cutting element is purposefully engaged when the local movement and the leading edge are aligned. This occurs once per single complete 360° local movement of the engagement element.

When the rotatable shaft is moving in a clockwise direction, then the effective cutting elements are most effective when they are biased to the “forward”/“clockwise” direction. When the rotatable shaft is placed in a reverse (anti-clockwise) direction, the orientation bias of the effective cutting elements is counterclockwise.

In a preferred embodiment, a bias of cutting elements designed to be facing in a clockwise direction, allows the back edge (non-cutting) of the engagement elements to be used as a compressing surface when the rotatable drive is placed in reverse. (Reverse) local and regional movements of the engagement elements will be now compress the swarf into the shaped surface of the contour body. The practical implication of this will be that the prepared surface of the work body now has a compacted/compressed shaped surface. In theory, this compaction of swarf results in a stronger, more resilient shaped surface so as to give additional strength to this surface.

Note that the independence of direction of the cutting elements (up/down/forward/back) is fundamentally different from the orientation of cutting elements (in a clockwise direction) of reamers of known art. As the local movements have a circular disposition, then any orientation of cutting element, will result in engagement of the cutting elements when the circular movement is aligned toward this direction.

Note also that in this embodiment, should the drive shaft rotation be placed in “reverse”, then the cutting elements will also be placed in a reverse movement pattern, allowing the non-cutting reverse aspects of the cutting elements to compress the contoured surface of the work body to give a compacted smooth finish to the surface.

It also demonstrates a spiral “down and forward” progression of cutting elements on the contour body. Note that this spiral or helical progression of cutting elements is an important part of the invention as it enables only a small part of the total cutting profile to be actively engaged at any particular time of the local movement pattern cycle. Cutting element 25, will be effectively engaged when the local movement pattern is aligned with this particular cutting element. This will be at a different time to the effective cutting time of element. This is important the mechanism results in low contact forces at any one time between contour body and work body.

### Concave/Convex

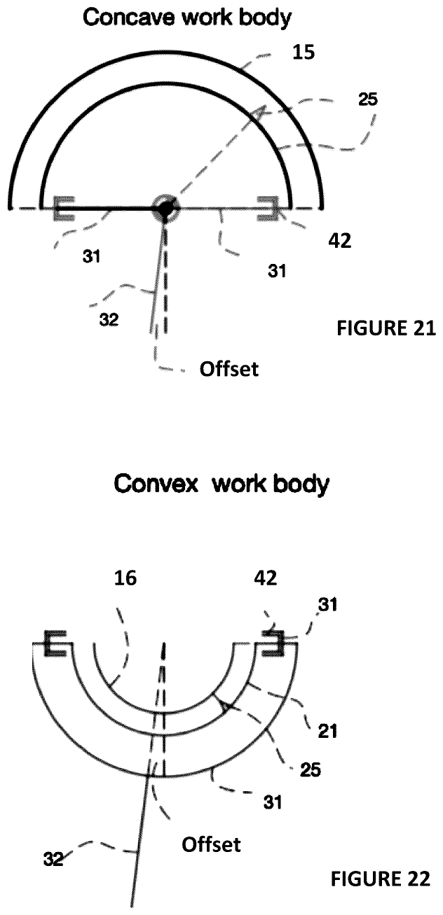

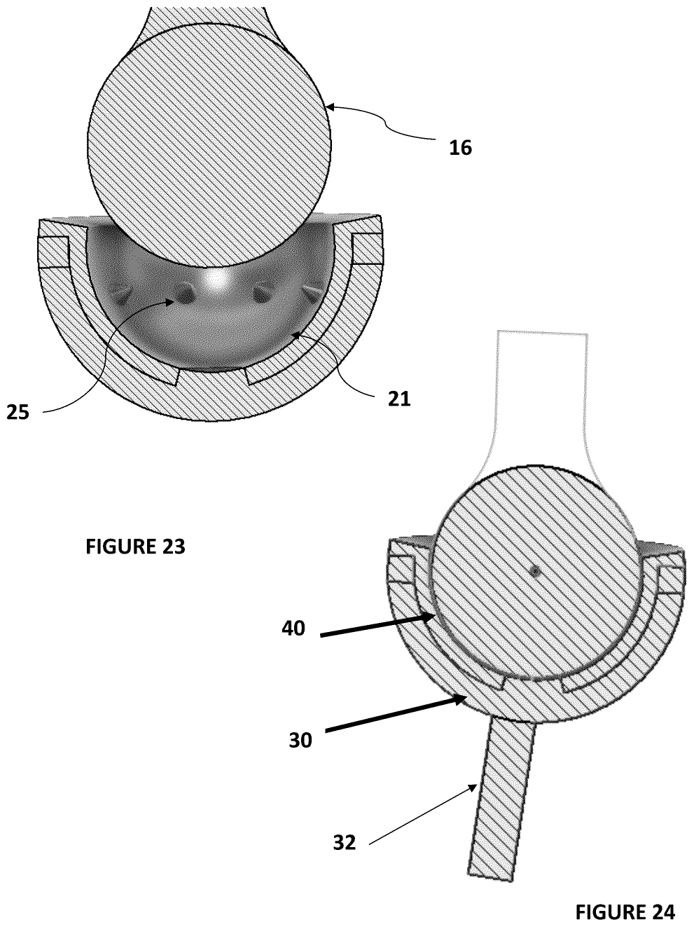

As shown in FIGS. 21 and 22, the shaping instrument 11 is not limited to the preparation of a concave inner surface 15 of a work body by a convex instrument. In a similar way the shaping instrument 11 can be for the preparation of a convex outer surface 16 of a work body by a concave instrument. What is applied to a concave inner surface 15 is similarly applied to a convex outer surface 16.

When the work body has a concave surface, then the outwardly protruding engagement elements 25 of the contour body project externally from the outer surface 21 of the contour body 20. When the work body has a convex surface 16, then the engagement elements 25 project internally from the inner surface 21 of the contoured body 20. A plurality of engagement elements 25 in various sizings and configurations can protrude outwardly from the outer surface 21 of the contour body 20 and be used to shape a concave surface 15 and similarly plurality of engagement elements 25 in various sizings and configurations can protrude inwardly from the inner surface 21 of the contour body 20 and be used to shape a convex surface 16.

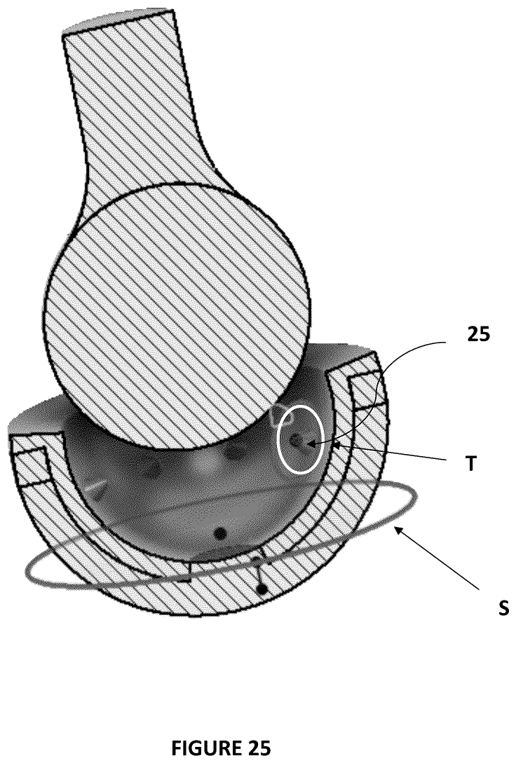

FIG. 25 demonstrate the movement patterns of the cutting (engagement) elements 25 of the contoured body when there is no frictional engagement of contoured body (regional movement), (S) and where there is frictional engagement of contour body (local movement), (T). This provides local and regional movement patterns of the engaging elements of the contour body.

### Symmetrical/Asymmetrical

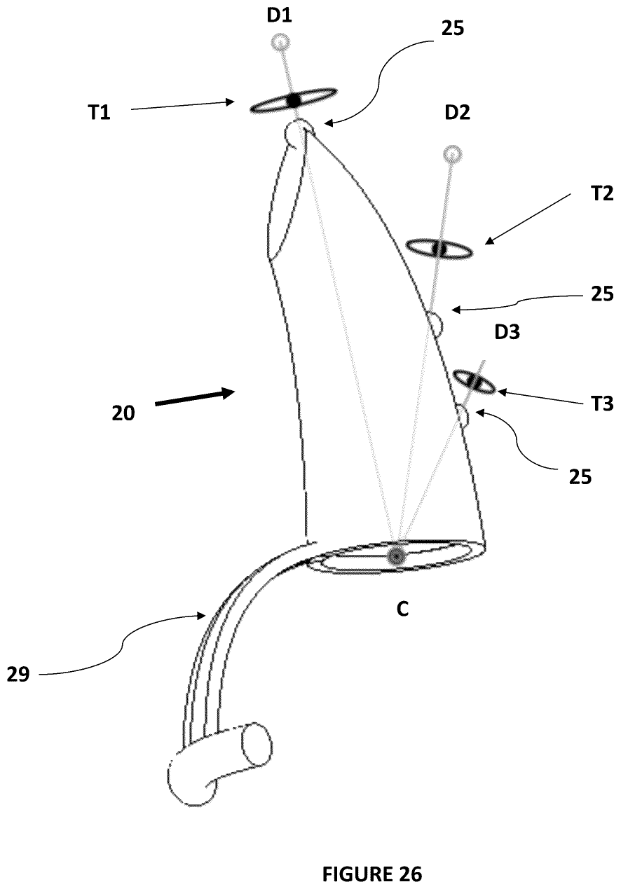

Similarly, as shown in FIG. 26 the shaping instrument 11 is not limited to a symmetrical shape but can be an asymmetrical shape which uses the same first and second transfer mechanisms 30, 40 to provide a localized effect T of the engagement elements 25. While the contour body of the symmetrical contour body 20 is generally “spherical” or “frusto-spherical”, the underlying mechanisms leading to “local” movement patterns can be applied to a non-hemispherical contour body. The contour body of the asymmetrical contour body 20 has a required shape relative to the work body with a plurality of engagement elements on the

The non-spherical contour body 20 is used to shape a work body in a non-spherical manner. When friction is applied to the contour body, the surface engagement elements 25 have local movement (T1, T2 or T3) around a radial axis (D1, D2 or D3) respectively leading to the Single Point Centre of Rotation (C).

There is a control handle 29 protruding from the non-hemispherical contour body 20. The handle 29 restricts “regional” circumferential movement and enables only small “local” movement patterns T1, T2 and T3 of engagement elements 25 placed on the outer surface of the contoured body 20.

In the use of this shaping instrument 11 in one preferred embodiment, the shaping instrument is used as a “starting router” prior to “first broach” in Total Hip Arthroplasty. This is a significant advancement to “known art” starting tools as the bone is occasionally hard and difficult to access with manual tools.

### Different Depths/Curvature

In order to further enhance capability of working on a work body the shaping instrument can be used in different forms and in different combinations.

In the embodiment of FIGS. 27 and 28, the shaping of a work body 15 can be considered as a one or two stage shaping process. In this preferred process, a two-step process is applied at different depths.

FIG. 27 demonstrates a non-cutting convex acetabular shell engaging with a concave work body (acetabulum). A standard reamer 55 acting as a depth “router” now protrudes from the acetabular shell 56, into the polar region of the work body 15 by a defined distance X. The “pilot hole” is thereby formed. Rotating the acetabular guide within the acetabulum, will engage the router to the known depth and in a direction determined by the surgeon. Thereby there is a polar contouring to a known depth and known direction as the “first stage” of the two-stage acetabular shaping process.

FIG. 28 demonstrate the resulting contouring of the first stage at the polar region 51 at the known depth X and known direction by the pilot hole “router”. In the second stage, once this pilot hole with known depth is prepared, the previously described acetabular reamer will advance until the “non-cutting” polar aspect of the reamer reaches the prepared floor. In this way, accidental excessive depth resection and wayward directional changes found in preparation of the acetabulum with traditional methodology will be effectively mitigated. Use of the work body 20 of the shaping instrument 11 without a polar cutting section, similar to the embodiment of FIG. 18 will result in controlled localized contouring around the engagement elements 25 in only the non-polar region 52.

A two-stage process, as described, for shaping the acetabulum during hip arthroplasty ensures a better safety and quality profile of acetabular preparation.

This two-stage or multi-stage process can be undertaken with different shaped and depth shaping instruments 11 to give control to differing locations and depths on the work body. Further the curvatures of the shaping instruments can be altered or used in different combinations to effect different effect on different angular surfaces of the work body. Still further as shown by the embodiment of FIG. 26 the combination could be included on a single non-symmetrical shaping instrument.

### Whole Contact/Localized Contact

The shaping instrument 11 of the invention has the substantial benefit of having engagement elements 25 that can be operable individually and locally. Control is by the contact of the particular engagement element 25 with the work body 15, 16.

When friction applied by the work body to the contour body is negligible, the second transfer mechanism 40 rotationally moves with the first transfer mechanism 30.

When contact increases friction, the second transfer mechanism 40 is coupled to the first transfer mechanism 30 and is a slave to all movements of the first transfer mechanism with the exception of rotation movement. The second transfer mechanism 40 is a coaxial annular plate 41 joined by bearing coupler 42 so it is able to independently rotate around its own longitudinal axis of rotation (A) as distinct to the drive angle of the longitudinal axis (B) of the drive shaft 32. Rotation R of the first transfer mechanism 30 around axis B of the and the slave movement of the second transfer mechanism 40 to the first transfer mechanism 30 with the exception of rotation movement results in the precessing movement T of the outwardly protruding engagement elements 25 around axis (D) on the surface of the convex curved body 20.

Therefore only one of the many engagement elements 25 of the contour body will be effectively engaged at any particular time. This is important as it means that the effective “working contact” between contour body and work body is small and therefore the drive mechanism is able to work at low torque and high rotation speeds.

### Use for Treatments

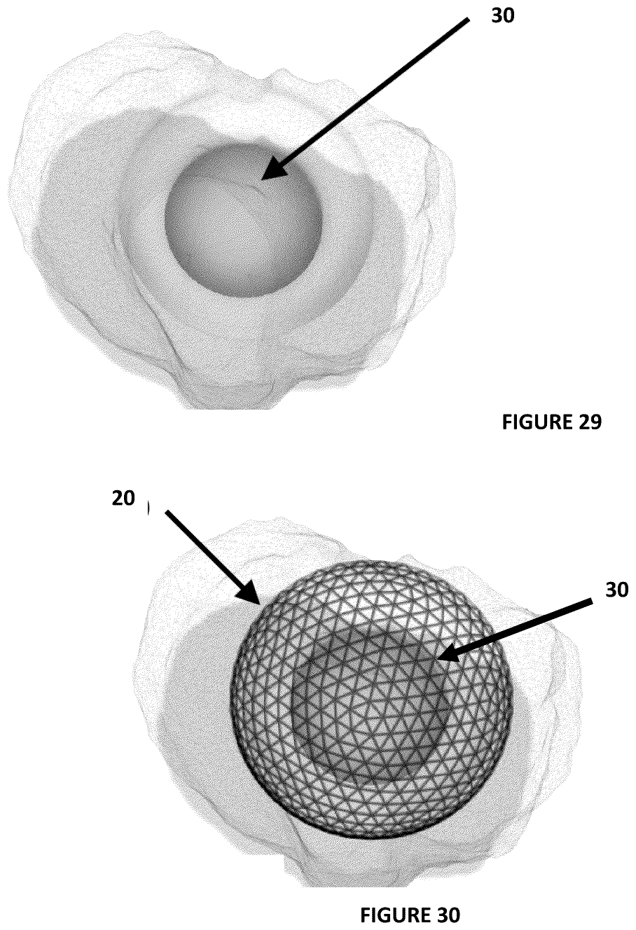

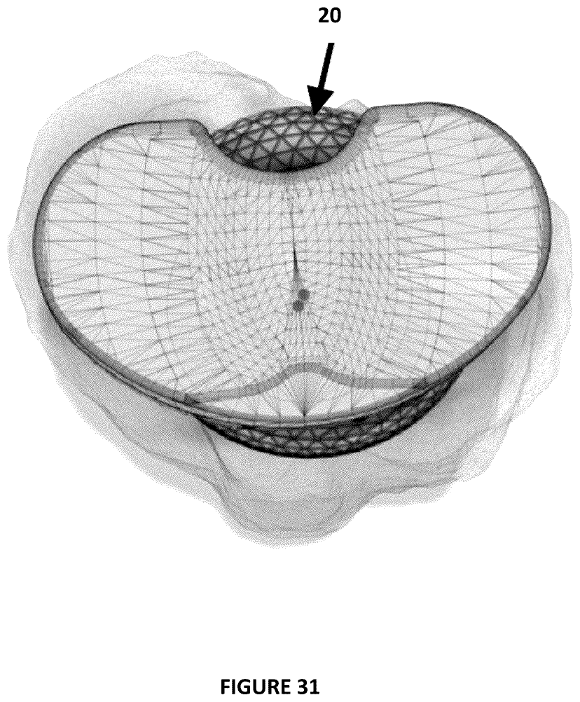

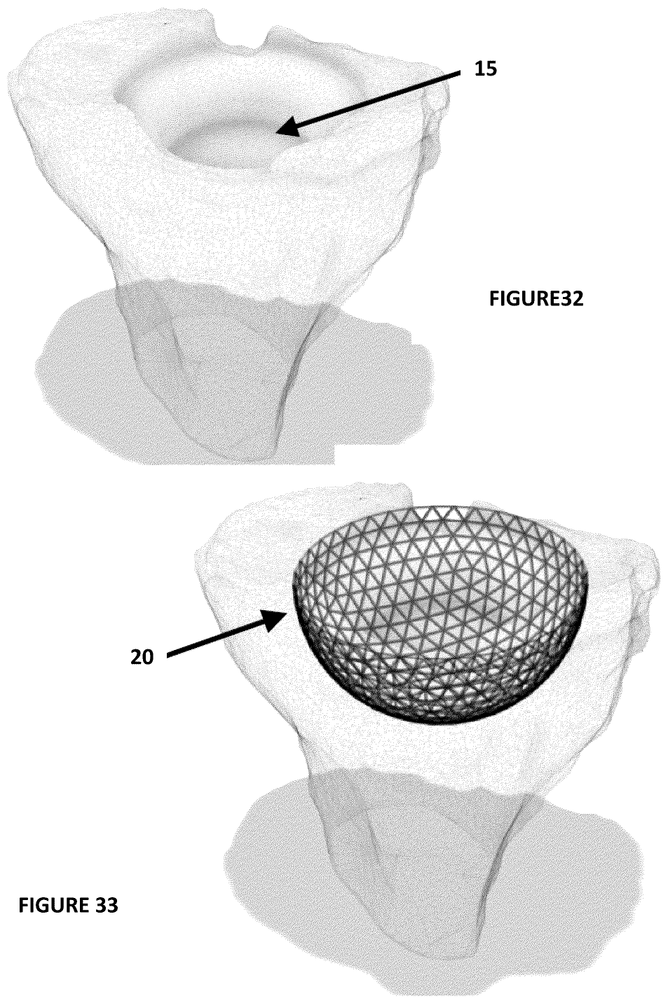

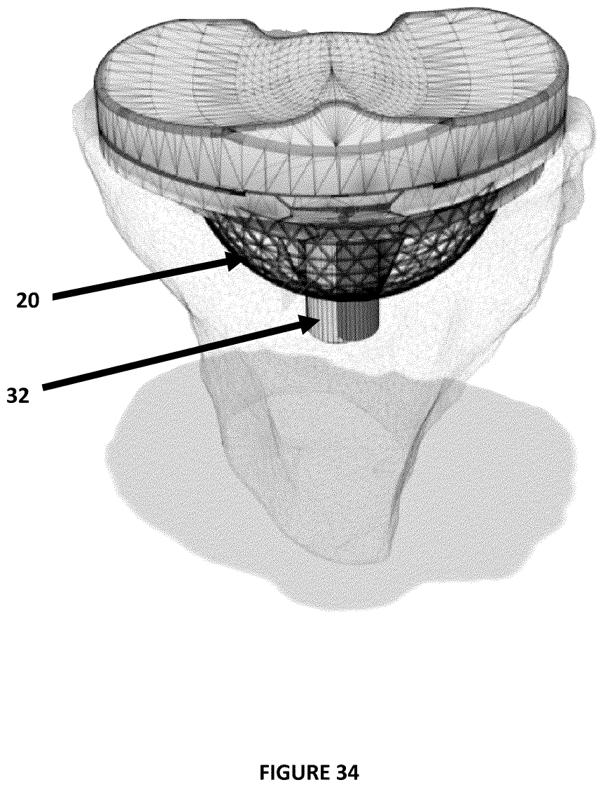

Further than the use of the invention for treatment for shaping the acetabulum during hip arthroplasty as shown in FIGS. 27, 28, there is shown in FIGS. 29 to 34 a specialized use for treatment where there is a proximal tibial bone defect. The work body 15 around the knee joint that has been shaped by contour body 20. However, in this form the same contour body can remain in the contoured bone and be used as a definitive structural support of the proximal tibial bone defect. The spherical contoured body can be used on the proximal tibial to support a proximal tibial bone defect and form a subsequent knee replacement.

However other uses of the invention are generally treatment comprising use of the shaping instrument 11 to contour the bone to allow for receipt of artificial bone supporting elements or artificial bone replacement joints that can be made of other materials.

As discussed, the shaping instrument 11 is not limited to one form. In one form the shaping instrument is a convex shaping instrument such as FIGS. 1 to 21, for use on a concave work body 15.

In a different embodiment, the shaping tool such as in FIGS. 22 to 25 can be used to shape bone as a convex work body 16, such as the femoral head during a “hip resurfacing” operation. The contour work body 16 is spherical and shaped to engage a convex surface such as the femoral head. The contour body 16 engages the concave curved body surface 21 with inwardly protruding engagement elements 25 which interfaces with second transfer mechanism 20 which interfaces with the first transfer mechanism 30 driven by the rotatable drive 32.

As shown in FIGS. 21 and 22, the comparison of treatment of convex shaping instrument 11 on concave work body 15 and concave shaping instrument 11 on a convex work body 16, FIG. 25 demonstrates a work body 16 such as a convex femoral head, into a concave contour body 20 with engagement elements 25. The same “local” (T) and “regional” (S) movement patterns of inwardly facing engagement elements 25 are effected where the single point centre of rotation of the contour body remains aligned with the longitudinal axis of the drive shaft 32.

It can be seen that a shaping instrument of the invention providing contouring is designed to shape or contour the underlying bone. This instrument cutting elements are designed to contour bone complementary to the movement output from the first and second transfer mechanisms.

The bone oscillating instrument can be used such as the asymmetrical form shown in FIG. 26 or the concave or convex forms comprising an inner and outer surface. The outer surface exhibits a number of elements for piercing, gouging, abrading or resecting bone. The wall of the instrument exhibits repeating inner structural voids that permit the passage of reamed bone from the outer to the inner shell surface. The internal architecture of the instrument can comprise additional structural elements used to compartmentalise bone graft generated from the bone reaming. The design allows the reamed or contoured autogenous bone to be trapped within the voids of the walls of the instrument, so forming an autogenous bone grafted instrument construct.

The shape of the reaming instrument is customised to the specific purpose required of the reamer. The instrument shape for surgery on the acetabulum during hip arthroplasty will be different from the instrument shape applied to the glenoid during shoulder arthroplasty or the instrument shape required to prepare a long bone for an intra-medullary device.

In one embodiment the instrument has a cutting zone and a non-cutting zone. The purpose of the non-cutting zone of the instrument allows for the regulation of direction and depth of bone contouring/shaping during bone preparation.

The primary function of the instrument is to shape/contour bone.

A second purpose of the instrument can be to act as structural support to the bone when bone deficiency is evident or where there is a requirement to stabilise one or more bones, with or without additional internal fixation. The seating of the instrument as a structural implant can follow its initial use as a contouring instrument when preparing the bone for the instrument definitive structural support. Indications for such use would include revision knee or hip surgery where volume bone voids of the femur, acetabulum or tibia are bridged by a site-specific instrument.

The function of the structural support can be enhanced, by designing structural compartments within the wall of the instrument, so that the host derived bone (autogenous graft) created during bone contouring can be incorporated into the wall of the instrument. In effect, this bone/instrument construct will be used as an as part of the definitive implant construct.

This feature can enhance bone ingrowth to the instrument acting as a structural support.

In addition to the reaming and the structural support functions, a third function or purpose of the instrument is to be used as an implantable prosthetic component. The instrument/prosthesis will typically contain compartmentalised autogenous host bone generated at the time of bone contouring.

The contouring instrument can be made from a range of material including, but not limited to titanium, chrome cobalt alloy, a plastic polymer or a combination of materials metal/plastic combination.

The contouring instrument can be single use or multiple use.

It can be a shaping instrument wherein the engagement elements are one or more of the type that:
a. Manipulate;
b. Sense;
c. Deform;
d. Cut;
e. Shave;
f. pierce,
g. gouge,
h. abrade or
i. resect.

The driving of the shaping instrument 11 is by a gearing mechanism that coverts uni-directional rotation movement of a Drill Driver to a drive shaft 32 to a multi-directional and multi-planar movement required of the instrument through a first transfer mechanism 30 and second transfer means 40 and the contoured body 20. The coupling to the second transfer mechanism 40 by the convex curved body 20 is in one form by attachment to the diametrically outer edges of the coaxial annular plate 41 of the second transfer mechanism 40.

The important element is that contoured body movement is a slave to the first transfer mechanism movement in all directions with the exception of rotation due to the rotation joint of the contact bearing 42 between first and second transfer mechanisms. Therefore the outwardly protruding engagement elements 25 precess around the axis of rotation D extending from the Single Point Centre Of Rotation (SPCOR) (C) and the axis of the outwardly protruding engagement elements 25. The result of rotation R of the first transfer mechanism 30 around axis B of the and the slave movement of the second transfer mechanism 40 to the first transfer mechanism 30 with the exception of rotation movement results in the precessing movement T of the outwardly protruding engagement elements 25 around axis (D) on the surface of the convex curved body 20.

Looking at the complex movement patterns, it can be seen that the transfer mechanisms convert the rotational movement of the rotatable drive 31 to at least one small local movement (T) of one or more parts 25 of the contoured body 20, wherein the shaping instrument 11 is able to work the work body surface by the at least one small local movement.

Rotational movement of the drive shaft 32 does not usually provide total rotational movement of the whole work body surface of the contoured curved body 20 such as movement (S), but provides a local movement pattern (T) of the of the outwardly protruding engagement elements 25 around their respective local axis D. This local movement pattern (T) prevails when friction is applied to the contoured/engagement elements 25 so driving the independent rotation between first and second transfer mechanisms 30, 40.

In one embodiment the invention can be used to create a spherical shape of known diameter into a third body with reference to a SPCOR. This can be achieved with rapid small regional movements of the surface of the second body against the third body.

In industry, this would allow the removal of material to create, re-furbish or recondition a spherical shape, without the need for circumferential movement of the tooling body around a longitudinal axis.

When applied to art of hip arthroplasty, the invention enables the contouring of a spherical acetabulum of a known size to accept a same sized spherical acetabular component. The contouring of a spherical acetabular shape using small regional movements of a multiple of single elements has safety advantages for both patient and surgeon. The existing prior art describes circumferential movement of contouring reamers around a longitudinal axis of drill or reamer. Such large-scale movement risks soft tissue injury. This risk is mitigated in the rapid small-scale movement of the invention described.

In another embodiment, the invention can be used to position a Third Body relative to a SPCOR so as to achieve a symmetrical positioning of a Third Body in relation to the SPCOR of a First or Second Body. Individual elements on a Second Body can influence the position of reference points on a Third Body such that the Third Body reference points are at the same distance to the SPCOR of a First or Second Body.

In a third embodiment, the invention would allow an assessment of forces of a third body with reference to the SPCOR of a First or Second Body. Second Body individual elements can be equipped with physical sensors that allow an assessment of the same physical parameters of an applied third body with reference to the SPCOR.

All pre-invention contouring bone reamers have a circular uni-directional rotational movement of the bone reamer. The reamer is described as rotating in a clockwise direction and the cutting aspects of the reamer are manufactured to cut/resect bone only when the reamer in moving in this clockwise direction.

### Interpretation

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but can. Furthermore, the particular features, structures or characteristics can be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention can be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### SCOPE OF INVENTION

Thus, while there has been described what are believed to be the preferred embodiments of the invention, those skilled in the art will recognize that other and further modifications can be made thereto without departing from the spirit of the invention, and it is intended to claim all such changes and modifications as fall within the scope of the invention. For example, any formulas given above are merely representative of procedures that can be used. Functionality can be added or deleted from the block diagrams and operations can be interchanged among functional blocks. Steps can be added or deleted to methods described within the scope of the present invention.

Although the invention has been described with reference to specific examples, it will be appreciated by those skilled in the art that the invention can be embodied in many other forms.

### INDUSTRIAL APPLICABILITY

It is apparent from the above, that the arrangements described are applicable to the material working industries which can include woodworking, arts, construction or medical and disability and prostheses industries.

1. A shaping instrument using regional or a combination of local and regional movement provided by a rotational drive means and one or more transfer means.

2. A shaping instrument according to claim 1 for the preparation of a surface of a work body including:
a. a contoured body having engagement elements on an active surface to work a passive surface of a work body;
b. a transfer mechanism connectable to the rotatable drive and engageable with the contoured body; and
wherein the contoured body and/or transfer mechanism are drivable by the rotational drive means.

3. A shaping instrument according to claim 2 wherein using a movement of local and/or regional movement the shaping instrument is able to work the passive surface of the work body by one or more of these two movements.

4. A shaping instrument according to claim 2 wherein linkage to the drive mechanism for regional movement is by direct linkage.

5. A shaping instrument according to claim 2 wherein the transfer mechanism converts the rotational movement of the rotatable drive to at least one small local movements of one or more parts of the contoured body or a circumferential regional movement of the same parts around a different (longitudinal) axis.

6. A shaping instrument according to claim 5 wherein the linkage to the drive mechanism for regional movement is by a semi linked clutch mechanism that uses frictional forces to advance the regional movement.

7. A shaping instrument according claim 2 wherein the drive mechanism for local movement is by the angled first transfer mechanism driving the second transfer mechanism in all directions except rotation around the axis of the rotatable drive.

8. A shaping instrument according to claim 7 wherein using only regional movement with depth control.

9. A shaping instrument according to claim 7 wherein the contoured body includes a non-cutting portion.

10. A shaping instrument according to claim 9 wherein the non-cutting portion of the contoured body is located at a polar top of the contoured body.

11. A shaping instrument according to claim 9 wherein the non-cutting portion of the contoured body of the shaping instrument is without a polar cutting section, and will result in controlled localized contouring around the engagement elements in only the non-polar region.

12. A shaping instrument according to claim 9 wherein the non-cutting portion regulates the direction and depth of bone resection.

13. A shaping instrument according to claim 2 wherein the rotational movement of a drive shaft provides total rotational movement of the whole work body surface of the contoured curved body.

14. A shaping instrument according to claim 2 wherein the shaping of a work body is a one or two stage shaping process.

15. A shaping instrument according to claim 14 wherein the shaping of a work body is a two-stage shaping process. wherein each of the two-step process is applied at a different depth.

16. A shaping instrument according to claim 14 wherein the two-stage or multi-stage process is undertaken with different shaped and depth shaping instruments to give control to differing locations and depths on the work body.

17. A shaping instrument according to claim 14 wherein different curvatures of the shaping instruments are used in different combinations to effect different effect on different angular surfaces of the work body.

18. A shaping instrument according to claim 2 wherein the local or regional movements are rotational movements around a respective radial axis to the single point centre of rotation (SPCOR) of the transfer mechanism.

19. A shaping instrument according to claim 18 wherein for the preparation of a surface of a work body including:
a. a contoured body having engagement elements on an active surface to work a passive surface of a work body;
b. a first transfer mechanism part connectable to a rotatable drive and including a rotationally offset base swash plate at a specified angle (defined 0°-180°) to the drive axis of the rotatable drive and rotatable with the rotatable drive for causing a precession or wobbling effect;
c. a second transfer mechanism part connectable to the contoured body and engageable with the first transfer mechanism part wherein any precession or wobbling effect of the first transfer mechanism part causes at least one small local movement of one or more parts of the contoured body in a rotational movement around a respective radial axis offset to the SPCOR located centrally in the second transfer mechanism; and
d. a second transfer mechanism part connectable to the contoured body and engageable with the first transfer mechanism part wherein the precession or wobbling effect of the first transfer mechanism part causes a regional circumferential rotation of one or more parts of the contoured body in a rotational movement around a longitudinal axis of the drive axis of the rotatable drive.

20. A shaping instrument according to claim 19 wherein the sizing of at least one small local movements (T) is dependent on one or more of:
a. the degree of the angular offset angle of the offset base swash plate offset angle to the drive axis of the rotatable drive;
b. the radial length of the contour body from a single point centre of rotation located centrally within the second transfer mechanism and aligned with the drive axis of the rotatable drive;
c. the linear translation (if any) between the Single point centre of Rotation and the longitudinal axis around which the rotatable drive rotates; and
d. the angulation (if any) between the axis around which the reamer rotates (on T1, the first transfer mechanism) and the axis around which T1 rotates in relation to T2 (the second transfer mechanism).
